(19) United States
(12) Patent Application Publication
(10) Pub. No.: US 2023/0366894 A1
(43) Pub. Date: Nov. 16, 2023

(54) CIRCULATING TOTAL-NT-PROBNP (GLYCOSYLATED AND UNGLYCOSYLATED NT-PROBNP) AND ITS RATIO WITH NT-PROBNP (UNGLYCOSYLATED NT-PROBNP) IN THE ASSESSMENT OF ATRIAL FIBRILLATION

(71) Applicant: Roche Diagnostics Operations, Inc., Indianapolis, IN (US)

(72) Inventors: Johann Karl, Penzberg (DE); Peter Kastner, Penzberg (DE); Roberto Latini, Milano (IT); Serge Masson, Rotkreuz (CH); Jennifer Marie Theresia Anna Meessen, Milano (IT); Vinzent Rolny, Penzberg (DE); Ursula-Henrike Wienhues-Thelen, Penzberg (DE); André Ziegler, Rotkreuz (CH)

(21) Appl. No.: 18/028,235

(22) PCT Filed: Sep. 30, 2021
(86) PCT No.: PCT/EP2021/076992

§ 371 Date: Mar. 24, 2023

(30) Foreign Application Priority Data
Sep. 30, 2020 (EP) 20199217.9

(51) Int. Cl. G01N 33/74

(52) U.S. Cl. CPC G01N 33/74; G01N 2333/58; G01N 2440/38; G01N 2800/326

(57) ABSTRACT

The present invention relates to a method for diagnosing atrial fibrillation in a subject, said method comprising the steps of a) determining the amount of total NT-proBNP in sample from the subject, b) determining the amount of unglycosylated NT-proBNP in a sample from the subject, c) calculating a score of the amounts determined in steps a) and b), d) comparing the calculated score with a reference score, and e) diagnosing atrial fibrillation in a subject.

### FIELD OF THE INVENTION

The present invention relates to a method for diagnosing atrial fibrillation in a subject, said method comprising the steps of a) determining the amount of total NT-proBNP in sample from the subject, b) determining the amount of unglycosylated NT-proBNP in a sample from the subject, c) calculating a score of the amounts determined in steps a) and b), d) comparing the calculated score with a reference score, and e) diagnosing atrial fibrillation in a subject.

### BACKGROUND OF THE INVENTION

Atrial fibrillation is the most common cardiac arrhythmia. However, atrial fibrillation (AF) is frequently not recognized by the patient. This is the case in approximately 40 % of patients indicating that history taking in insensitive for the diagnosis of atrial fibrillation (Kamel H. et al, Curr Atheroscler Rep 2011: 13: 338 - 343).

The gold standard to detect atrial fibrillation is the electrocardiogram (ECG), preferably performed as 24 hour ECG (Holter Monitoring). However, Holter Monitoring can only detect atrial fibrillation if the arrhythmia occurs in the 24 hour period of ECG recording.

Several publications implicate the association of biomarkers at enhanced levels with atrial fibrillation (Biomarkers in Atrial Fibrillation: Investigating Biologic Plausibility, Cause, and Effect R. Becker Journal of Thrombosis and Thrombolysis 19(1), 71-75, 2005).

For example, Buettner et al. investigates the association between N-terminal (NT)-proBNP and NT-proANP levels with 3 Atrial Fibrillation (AF) progression phenotypes. It was shown that natriuretic peptides show different sensitivity for phenotypes of AF progression (Role of NT-proANP and NT-proBNP in patients with atrial fibrillation: Association with atrial fibrillation progression phenotypes, Büttner, Petra et al. Heart Rhythm, Volume 15, Issue 8, 1132 - 1137).

WO 2014/072500 discloses that NT-proBNP can be used for the assessment of a recent atrial fibrillation.

Chang et al. disclose the use of NTproBNP (and other biomarkers) in the assessment of atrial fibrillation (Afib): BNP and NT-proBNP have been shown to be associated with atrial fibrillation, incidence, postoperative atrial fibrillation incidence, and prognosis in atrial fibrillation (Chang et al. Clinical Applications of Biomarkers in Atrial Fibrillation, The American Journal of Medicine, Vol 130, No 12, December 2017).

Brain natriuretic peptide (BNP) is a 32-amino acid polypeptide. BNP is synthesized as a 134-amino acid pre-prohormone (“pre-proBNP”). Removal of the N-terminal signal peptide which has a length of 26 amino acids generates the prohormone (“proBNP”, 108 aa long). The prohormone is subsequently cleaved into NT-proBNP (N-terminal of the prohormone brain natriuretic peptide, 76 aa long) and the biologically active brain natriuretic peptide (BNP). NT-proBNP and BNP are produced in equimolar amounts. Several studies showed that assays for BNP and NT-proBNP can be reliably used for the diagnosis of heart failure (see e.g. Prontera et al., Clinica Chimica Acta 400 (2009) 70-73).

B-type natriuretic peptide (BNP) and N-terminal proBNP (NT-proBNP) are peptides produced in the heart in response to increased wall stretch and volume overload (see e.g. Semenow et al., Clinical Chemistry 65:9 (2019) 1070-72).

Schellenberger et al. reported that the precursor to B-type natriuretic peptide is an O-linked glycoprotein (Schellenberger et al., Arch Biochem Biophys 2006, 451). To date, there are nine known O-glycosylation sites on proBNP and NTproBNP (Halfinger et al: Unraveling the Molecular Complexity of O-Glycosylated Endogenous (N-Terminal) pro-B-TypeNatriuretic Peptide Forms in Blood Plasma of Patients with Severe Heart Failure. Clinical Chemistry 63:1, 359-368 (2017)).

Recently, proBNP glycosylation has emerged as a potential regulatory mechanism in the production of amino-terminal (NT)-proBNP and BNP (see e.g. Vodovar et al., European Heart Journal, Volume 35, Issue 48, 21 Dec. 2014, Pages 3434-3441). Among the 9 identified glycan attachment sites within a proBNP molecule, glycosylation of the region close to the proBNP cleavage site was shown to play a pivotal role in regulating the enzyme-mediated processing of proBNP (Semenow et al., Clinical Chemistry 65:9 (2019) 1071). Patients with chronic HF had the highest percentage of glycosylated proBNP as compared to patients with acute decompensated HF and non-acute decompensated HF (see e.g. Vodovar et al). Semenow et al. assumes that that in chronic HF increased release of glycosylated proBNP that is not sufficiently processed into BNP will lead to a comparably low amount of the active hormone, despite high measured concentrations of proBNP

Røtjø et al examined the influence of glycosylation on the diagnostic and prognostic accuracy of NT-proBNP in unselected patients with dyspnea and found higher NT-proBNP concentrations after removing sugar moieties from NTproBNP1-76 by the use of deglycosylation enzymes in plasma samples from patients presenting with dyspnea, particularly in those with confirmed heart failure. (Helge Røsjø et al: Influence of Glycosylation on Diagnostic and Prognostic Accuracy of N-Terminal Pro-B-Type Natriuretic Peptide in Acute Dyspnea: Data from the Akershus Cardiac Examination 2 Clinical Chemistry 61:8, 1087-1097 (2015).

In the study of Lewis et al., assays were developed to distinguish between total proBNP (glycosylated plus nonglycosylated proBNP), proBNP not glycosylated at threonine 71, and proBNP not glycosylated in the central region. It was shown that proBNP that is not glycosylated at threonine 71 is decreased with obesity in patients with heart failure (Semenow et al., 2019; Lewis et al., Clin Chem 2019;65:1115-24).

So far, it has not been assessed whether glycosylation of NT-proBNP (or its precursor proBNP) plays a role in atrial fibrillation. Advantageously, it was found in the studies underlying the present invention that the determination of both total NT-proBNP and unglycosylated NT-proBNP, and the calculation of a score based on the amounts of total NT-proBNP and unglycosylated NT-proBNP (such as a ratio) allows for a reliable assessment of atrial fibrillation. Interestingly, the assessment based on the two biomarkers, i.e. total NT-proBNP and unglycosylated NT-proBNP is better than the assessment based on the biomarkers when determined alone.

Accordingly, the present invention relates to a method for diagnosing atrial fibrillation in a subject, said method comprising the steps of
a) determining the amount of total NT-proBNP in sample from the subject,
b) determining the amount of unglycosylated NT-proBNP in a sample from the subject,
c) calculating a score of the amounts determined in steps a) and b), and
d) comparing the calculated score with a reference score.

In some embodiments, the method of the present invention comprises the further step of

e) diagnosing atrial fibrillation in a subject.

Preferably, step e) is based on the results of the comparison step d). Accordingly, step e) may be as follows:

e) diagnosing atrial fibrillation in a subject based on the results of the comparison in step d).

The method as referred to in accordance with the present invention includes a method which essentially consists of the aforementioned steps or a method which includes further steps. Moreover, the method of the present invention, preferably, is an ex vivo and more preferably an in vitro method. Moreover, it may comprise steps in addition to those explicitly mentioned above. For example, further steps may relate to the determination of further marker and/or to sample pre-treatments or evaluation of the results obtained by the method. The method may be carried out manually or assisted by automation. Preferably, step (a), (b), (c), (d) and/or (e) may in total or in part be assisted by automation, e.g., by a suitable robotic and sensory equipment for the determination in step (a) and (b) or a computer-implemented calculation in step (c) or a computer-implemented comparison in step (d). Thus, the methods of the present invention may be computer-implemented

The term “diagnosing” as used herein, preferably, means assessing whether a subject as referred to in accordance with the method of the present invention suffers from atrial fibrillation (AF), or not. Preferably, the expression “diagnosing atrial fibrillation” as used herein shall be understood as “aiding” or “assisting” in the diagnosis of atrial fibrillation. For example, a physician may be assisted in the diagnosis of atrial fibrillation by additional information and/or devices. Thus, the actual diagnosis might be carried out by a physician.

As will be understood by those skilled in the art, the diagnosis of the present invention is usually not intended to be correct for 100% of the subjects to be tested. The term “diagnosing”, preferably, requires that a correct diagnosis can be made for a statistically significant portion of subjects. Whether a portion is statistically significant can be determined without further ado by the person skilled in the art using various well known statistic evaluation tools, e.g., determination of confidence intervals, p-value determination, Students t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.4, 0.1, 0.05, 0.01, 0.005, or 0.0001.

The method of the present invention shall aid in the diagnosis of atrial fibrillation. The term “atrial fibrillation” (“abbreviated” AF or AFib) is well known in the art. As used herein, the term preferably refers to a supraventricular tachyarrhythmia characterized by uncoordinated atrial activation with consequent deterioration of atrial mechanical function. In particular, the term refers to an abnormal heart rhythm characterized by rapid and irregular beating. It involves the two upper chambers of the heart. In a normal heart rhythm, the impulse generated by the sino-atrial node spreads through the heart and causes contraction of the heart muscle and pumping of blood. In atrial fibrillation, the regular electrical impulses of the sino-atrial node are replaced by disorganized, rapid electrical impulses which result in irregular heart beats and increased atrial volume. The increased volume leads to stretch of the tissue that releases natriuretic peptides. Symptoms of atrial fibrillation are heart palpitations, fainting, shortness of breath, or chest pain. However, many episodes have no symptoms. On the electrocardiogram (ECG), Atrial Fibrillation is characterized by the replacement of consistent P waves by rapid oscillations or fibrillatory waves that vary in amplitude, shape, and timing, associated with an irregular, frequently rapid ventricular response when atrioventricular conduction is intact.

The European Society of Cardiology (ESC) and propose the following classification system (see Hindricks G et al, doi:10.1093/eurheartj/ehaa612; which herewith is incorporated by reference in its entirety, see e.g Table 4 in the cited document): First diagnosed AF, paroxysmal AF, persistent AF, long-standing persistent and permanent AF.

All people with AF are initially in the category called first diagnosed AF. However, the subject may or may not have had previous undetected episodes. A subject suffers from permanent AF, if the AF has persisted for more than one year and it is accepted by the patient and physician, that no further attempts to restore/maintain sinus rhythm will be undertaken. In particular, conversion back to sinus rhythm does not occur (or only with medical intervention). A subject suffers from persistent AF, if the AF lasts more than 7 days. The subject may require either pharmacologic or electrical intervention to terminate Atrial Fibrillation. Thus persistent AF occurs in episodes, but the arrhythmia does not convert back to sinus rhythm spontaneously. Paroxysmal Atrial Fibrillation, preferably, refers to an intermittent episode of Atrial Fibrillation that terminates spontaneously (or with intervention) within 7 days of onset. In most cases of paroxysmal AF, the episodes last less than 24 hours The episodes of paroxysmal Atrial Fibrillation terminate often spontaneously, i.e. without medical intervention. Paroxysmal AF is often asymptomatic and underdiagnosed (silent AF). A preferred episode length of the present invention is shorter than 48 hrs, 24 hrs or 12 hrs (paroxysmal AF) Accordingly, the term “paroxysmal atrial fibrillation” as used herein is defined as episodes of AF that self-terminate, preferably, in less than 48 hours, more preferably in less than 24 hours, and, most preferably in less than 12 hours. Preferably, said episodes are recurrent. Further, it is envisaged that the episodes self-terminate in less than 6 hours.

Both persistent and paroxysmal AF may be recurrent within weeks or months, whereby distinction of paroxysmal and persistent AF is provided by ECG recordings: When a patient has had two or more episodes, AF is considered recurrent If the arrhythmia terminates spontaneously, AF, in particular recurrent AF, is designated paroxysmal. AF is designated persistent if it lasts more than 7 days.

In some embodiments of the method of the present invention, the atrial fibrillation to be diagnosed is paroxysmal or persistent atrial fibrillation. In some embodiments of the method, the atrial fibrillation is persistent atrial fibrillation.

In accordance with the present invention, it is envisaged that the atrial fibrillation to be diagnosed is an ongoing atrial fibrillation Thus, a subject who suffers from atrial fibrillation exhibits an episode of atrial fibrillation at the time of the testing (or to be more precise at the time at which the test sample has been obtained).

The term “sample” refers to a sample of a body fluid, to a sample of separated cells or to a sample from a tissue or an organ. Samples of body fluids can be obtained by well-known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

In some embodiments of the method of the present invention, the sample is blood sample (i.e a whole blood sample), a serum sample, or a plasma sample.

In some embodiments of the methods of the present invention, the sample is a right atrial appendage tissue sample

The term “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the subject is a human. The subject can be male or female. The terms “patient” and “subject” are used interchangeably herein.

In an embodiment, the subject is a female subject. In another embodiment, the subject is a male subject.

In some embodiments, the subject may have at least one risk factor for atrial fibrillation, such as hypertension, such as hypertension requiring anti-hypertensive medication, heart failure, such as heart failure AHA stage A - C, history of stroke.

In some embodiments, the subject to be tested is 50 years of age or older, such as 60 years of age or older. In some embodiments, the subject is 70 years of age or older.

Preferably, the subject to be tested is a subject who is suspected to suffer from atrial fibrillation. A subject who is suspected to suffer from atrial fibrillation is a subject who shows at least one symptom of atrial fibrillation and/or who has shown at least one symptom of atrial fibrillation prior to carrying out the method for assessing atrial fibrillation Said symptoms are usually transient and may arise in a few seconds and may disappear just as quickly. Symptoms of atrial fibrillation include dizziness, fainting, shortness of breath and, in particular, heart palpitations. Accordingly, the at least one symptom of atrial fibrillation is selected from dizziness, fainting, shortness of breath and, in particular, heart palpitations. Preferably, the subject has shown at least one symptom of atrial fibrillation within six months, more preferably within one month, even more preferably within two weeks, and most preferably within one week prior to obtaining the sample. In particular, it is envisaged that the subject has shown at least one symptom of AF within 2 days prior to obtaining the sample. Also, it is envisaged that the subject has shown at least one symptom of AF within 24 hours, or even within 12 hours prior to obtaining the sample. Thus, the subject is suspected to have exhibited an episode of atrial fibrillation, i.e. atrial fibrillation, within one of these periods

In accordance with the present invention it is envisaged that the subject who is suspected to suffer from atrial fibrillation has a history of atrial fibrillation, i.e. a known history of atrial fibrillation. Accordingly, the subject shall have been diagnosed to suffer from atrial fibrillation previously, i.e. before carrying out the method of the present invention (in particular before obtaining the sample from the subject). In addition, the subject may have had previous undiagnosed episodes of atrial fibrillation.

According to step a) of the method of the present invention, the amount of total NT-proBNP is determined, i.e. measured, in sample from the subject. According to step b), the amount of unglycosylated NT-proBNP is determined in a sample, such as a blood, serum or plasma sample, from the subject. It is to be understood that steps a) and b) can be carried out in any order. Further, the steps may be carried out simultaneously.

The term “NT-proBNP” (N-terminal fragment of pro-brain natriuretic peptide) is well known in the art. As used herein, the term relates to the 76 amino acid N-terminal fragment of pro brain natriuretic peptide (proBNP) which is a secreted protein which, after cleavage, functions as a cardiac hormone. Methods to measure NT-proBNP are commercialized and used in clinical practice such as distributed under the name Roche Elecsys® proBNP II.

The sequence of human NT-proBNP is well known in the art and has been described already in detail in the prior art, e.g., WO 02/089657, WO 02/083913, Bonow 1996, New Insights into the cardiac natriuretic peptides. Circulation 93: 1946-1950. Preferably, human NT-proBNP has an amino acid sequence as shown in SEQ ID NO. 1.

As described above, NT-proBNP as well as its precursor proBNP can be O-glycosylated. An overview is on the O-Glycosylation of NT-proBNP and proBNP is, e.g., provided in by Schellenberger et al. and Halflinger et al. which are both incorporated by reference with respect to their entire disclosure content (Schellenberger et al., Arch Biochem Biophys 2006; 451; Halfinger et al., Clinical Chemistry 63:1, 359-368 (2017).

The term “O-linked glycosylation” is well known in the art (herein also referred to as “glycosylation”). O-linked glycosylation is the attachment of a sugar molecule, i e a carbohydrate to serine or threonine residues. It is a post-translational modification that occurs after the protein has been synthesized. How NT-proBNP and proBNP are glycosylated is, for example, described in Schellenberger et al. and Halflinger et al. (cited above).

An overview on O-Olycosylation sites of NT-proBNP is provided herein below. The following sequence is the human NT-proBNP sequence (SEQ ID NO. 1 which serves as a reference sequence here). The O-Glycosylation sites are underlined:

His Pro Leu Gly Ser Pro Gly Ser Ala Ser Asp Leu Glu Thr Ser Gly
1 5 10 15
Leu Gln Glu Gln Arg Asn His Leu Gln Gly Lys Leu Ser Glu Leu Glu
20 25 30
Val Glu Gln Thr Ser Leu Glu Pro Leu Gln Glu Ser Pro Arg Pro Thr
35 40 45
Gly Val Trp Lys Ser Arg Glu Val Ala Thr Glu Gly Ile Arg Gly His
50 55 60
Arg Lys Met Val Leu Tyr Thr Leu Arg Ala Pro Arg (SEQ ID No: 1)
65 70 75

Thus, human NT-proBNP (as shown in SEQ ID NO: 1) comprises at least the following glycosylation sites: T36, S37, S44, T48, S53, T58 and T71.

Preferably, the term “unglycosylated NT-proBNP” as used herein, refers to NT-proBNP which is not glycosylated (i.e. which is not O-glycosylated) at one or more positions selected from group consisting of T36, S37, S44, T48, S53, T58 and T71 of human NT-proBNP. Thus, at least one of the following amino acid residues of human NT-proBNP is not glycosylated, i.e. does not comprise an O-glycosylation: T36, S37, S44, T48, S53, T58 and T71.

In some embodiments, the term “unglycosylated NT-proBNP”, refers to NTproBNP in which at least two of the aforementioned amino acid residues (i e. of T36, S37, S44, T48, S53, T58 and T71) are not glycosylated. In some embodiments, the term “unglycosylated NT-proBNP”, refers to NTproBNP in which at least three of the aforementioned amino acid residues are not glycosylated. In some embodiments, the term “unglycosylated NT-proBNP”, refers to NTproBNP in which at least three, at least four, at least five, at least six, or all of the aforementioned amino acid residues are not glycosylated. In some embodiments, the term “unglycosylated NT-proBNP”, refers to NTproBNP in which at least the serine residue at position 44 (i.e. S44) is not glycosylated Thus, unglycosylated NT-proBNP may be unglycosylated at position S44 (i.e. Ser 44).

In a preferred embodiment, determining of the amount of unglycosylated NT-proBNP in a sample from the subject comprises contacting the sample with an antibody, or antigen-binding fragment thereof, which specifically detects unglycosylated NT-proBNP. Preferably, the antibody, or antigen-binding fragment thereof, which specifically detects unglycosylated NT-proBNP specifically binds to an epitope of NT-proBNP which epitope comprises a glycosylation site, but which is not glycosylated at the glycosysation site. The formed complex between the antibody (or fragment) and the biomarker shall be proportional to the amount of the unglycosylated NT-proBNP.

In some embodiments, the antibody, or antigen-binding fragment thereof, which specifically detects unglycosylated NT-proBNP specifically binds to an epitope of NT-proBNP which epitope comprises the T36 amino acid residue, wherein said T36 amino acid residue is not glycosylated. Preferably, said antibody or fragment essentially does not bind NT-proBNP comprising a glycosylated T36 amino acid residue.

In some embodiments, said antibody, or fragment thereof, specifically binds to an epitope of NT-proBNP which epitope comprises S37 amino acid residue, wherein said S37 amino acid residue is not glycosylated. Preferably, said antibody or fragment essentially does not bind NT-proBNP comprising a glycosylated S37 amino acid residue.

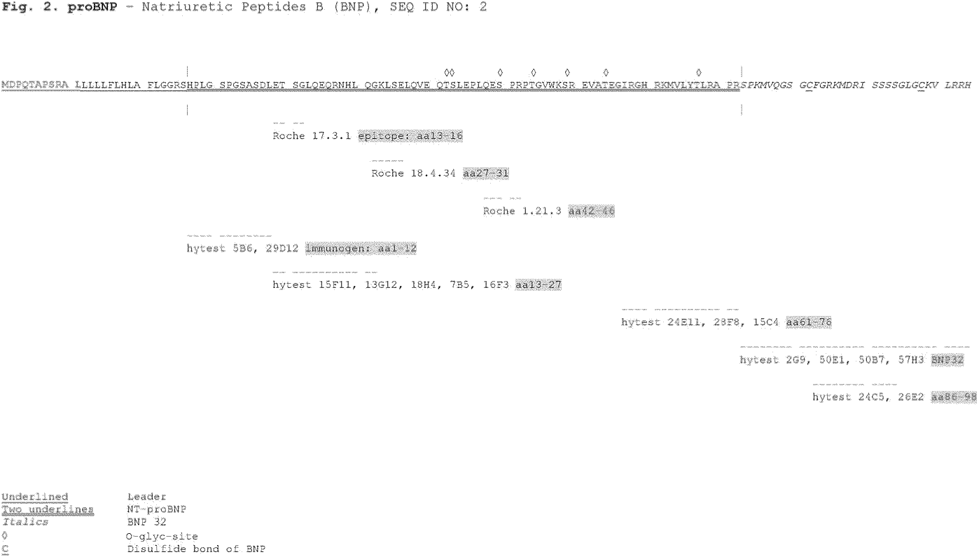

In some embodiments, said antibody, or fragment thereof, specifically binds to an epitope of NT-proBNP which epitope comprises the S44 amino acid residue, wherein said S44 amino acid residue is not glycosylated. Preferably, said antibody or fragment essentially does not bind NT-proBNP comprising a glycosylated S44 amino acid residue. In some embodiments, the epitope of said antibody, or antigen-binding fragment thereof, comprises amino acid residues 42 to 46 of human NT-proBNP (as shown in SEQ ID NO: 1, see also FIG. 2).

In a preferred embodiment, the antibody which specifically detects unglycosylated NT-proBNP is the monoclonal antibody MAB 1.21.3 as disclosed in WO2004099253A1, or an antibody, which comprises the six CDRs of said antibody. Further, it is envisaged to use an antigen-binding fragment of said antibody

In some embodiments, said antibody, or fragment thereof, specifically binds to an epitope of NT-proBNP which epitope comprises the T48 amino acid residue, wherein said T48 amino acid residue is not glycosylated. Preferably, said antibody or fragment essentially does not bind NT-proBNP comprising a glycosylated T48 amino acid residue

In some embodiments, said antibody, or fragment thereof, specifically binds to an epitope of NT-proBNP which epitope comprises the S53 amino acid residue, wherein said S53 amino acid residue is not glycosylated Preferably, said antibody or fragment essentially does not bind NT-proBNP comprising a glycosylated S53 amino acid residue.

In some embodiments, said antibody, or fragment thereof, specifically binds to an epitope of NT-proBNP which epitope comprises the T58 amino acid residue, wherein said T58 amino acid residue is not glycosylated Preferably, said antibody or fragment essentially does not bind NT-proBNP comprising a glycosylated T58 amino acid residue.

In some embodiments, said antibody, or fragment thereof, specifically binds to an epitope of NT-proBNP which epitope comprises the T71 amino acid residue, wherein said T71 amino acid residue is not glycosylated Preferably, said antibody or fragment essentially does not bind NT-proBNP comprising a glycosylated T71 amino acid residue.

Preferably, the term “glycosylated NT-proBNP” as used herein, preferably, refers to NT-proBNP which glycosylated (i.e. O-glycosylated) at one or more positions (i.e. amino acid residues) selected from group consisting of T36, S37, S44, T48, S53, T58 and T71 of human NT-proBNP. Thus, at least one of the following amino acid residues of human NT-proBNP is glycosylated, i.e. comprises an O-glycosylation: T36, S37, S44, T48, S53, T58 and T71.

Further, suitable epitopes are disclosed in FIG. 2.

In some embodiments, the term “glycosylated NT-proBNP”, refers to NTproBNP in which at least two of the aforementioned amino acid residues (i.e. of T36, S37, S44, T48, S53, T58 and T71) are glycosylated. In some embodiments, the term “glycosylated NT-proBNP”, refers to NTproBNP in which at least three of the aforementioned amino acid residues are glycosylated. In some embodiments, the term “glycosylated NT-proBNP”, refers to NTproBNP in which at least three, at least four, at least five, at least six, or all of the aforementioned amino acid residues are glycosylated. In some embodiments, the term “glycosylated NT-proBNP”, refers to NTproBNP in which at least the serine residue at position 44 (i e. S44) is glycosylated.

The “total amount of NT-proBNP” is preferably the amount of glycosylated and unglycosylated NT-proBNP The term, thus, refers to the sum of the amount of glycosylated NT-proBNP and unglycosylated NT-proBNP.

Preferably, the determination of the amount of total NT-proBNP comprises contacting the sample with an antibody, or antigen-binding fragment thereof, which specifically detects total NT-proBNP. More preferably, said antibody, or antigen-binding fragment thereof, which specifically detects total NT-proBNP binds to a region of human NT-proBNP which can not be glycosylated. Accordingly, said antibody (or fragment thereof) shall specifically bind to a region of NT-proBNP, in particular of human NT-proBNP, which does not carry a glycosylation site, i.e. an O-glycosylation site. The formed complex between the antibody (or fragment) and the biomarker shall be proportional to the amount of the total NT-proBNP.

In particular, said antibody (or fragment thereof) shall specifically bind to a region of NT-proBNP which does not carry the T36, S37, S44, T48, 553, T58 or T71 glycosylation site (of human NT-proBNP). For example, the first 35 amino acid residues, i.e. the N-terminal amino acid residues 1 to 35 are known to carry no O-glycosylation sites. Preferably, the antibody, or antigen-binding fragment thereof, which specifically detects total NT-proBNP binds to an epitope present within the first 35 amino acids of NT-proBNP, more preferably, it binds to an epitope present within the first 20 amino acids of NT-proBNP, most preferably, it binds to an epitope present within amino acids residues 10 to 20 of human NT-proBNP. In a preferred embodiment, the epitope of said antibody, or antigen-binding fragment thereof, comprises amino acid residues 13 to 16 of human NT-proBNP. The sequence of human NT-proBNP is shown above (see SEQ ID NO. 1).

In a preferred embodiment, the antibody which specifically detects total NT-proBNP is the monoclonal antibody MAB 17.3.1 as disclosed in WO2004099253A1, or an antibody, which comprises the six CDRs of said antibody. Further, it is envisaged to use an antigen-binding fragment of said antibody.

The term “antibody” is known in the art As used herein, the term refers to any immunoglobulin (Ig) molecule comprised of four polypeptide chains, two heavy (H) chains and two light (L) chains. As used herein, the term “antibody” also includes an antigen-binding fragment of the antibody. As used herein, an antigen-binding fragment of an antibody shall be capable of specifically binding to the antigen (in particular to the NT-proBNP as described above. Thus, antigen binding fragments of antibodies are fragments retaining the ability of the (full-length) antibody to specifically bind to the antigen (such as unglycosylated NT-proBNP or total NT-proBNP). Antibody fragments preferably comprise a portion of a full length antibody, preferably the variable domain thereof, or at least the antigen binding site thereof In an embodiment, the antigen-binding fragment is selected from the group consisting of a Fab fragment, a Fab′ fragment, a Facb fragment, a F(ab′)2 fragment, a scFv fragment, an a Fv fragment. For example, the antigen-binding fragment is a F(ab′)2 fragment. How to produce antigen-binding fragments is well known in the art. For example, the fragments can be produced by enzymatic cleavage of an antibody of the present invention. In addition, the fragments can be generated by synthetic or recombinant techniques. Fab fragments are preferably generated by papain digestion of an antibody, Fab′ fragments by pepsin digestion and partial reduction, F(ab′)2 fragments by pepsin digestion), and facb fragments by plasmin digestion. Fv or scFv fragments are preferably produced by molecular biology techniques.

The antibody in accordance with the method of the present invention can be a polyclonal or monoclonal antibody. In a preferred embodiment, the antibody is a monoclonal antibody. The term “monoclonal antibody” is well known in the art. As used herein, the term preferably refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. A monoclonal antibody of the present invention can be made by the well-known hybridoma method described by Kohler and Milstein, Nature, 256:495 (1975), or can be made by recombinant DNA methods. In some embodiments, the monoclonal antibody is selected from a group consisting of a sheep monoclonal antibody, a mouse monoclonal antibody, a rabbit monoclonal anti-body, a goat monoclonal antibody, a horse monoclonal antibody, a chicken monoclonal antibody. In some embodiments, the monoclonal antibody is a mouse monoclonal antibody.

The antibodies (or fragments) that are used in steps a) and b) of the method of the present invention can be used in a sandwich assay as capture antibody in combination with at least one other antibody binding to a different, i e further NT-proBNP epitope. Preferably, said at least one other antibody binds to a region of NT-proBNP which is not glycosylated as described above.

The antibodies can be used in sandwich assays. Sandwich assays are among the most useful and commonly used assays encompassing a number of variations of the sandwich assay technique. For example, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent Any un-reacted material may be washed away, and the presence of the biomarker is determined by observation of a signal produced by the reporter molecule bound to the detection binding agent The results may either be qualitative, by simple observation of a visible signal, or may be quantitated by comparison with a control sample containing e.g. known amounts of the biomarker to be determined (as standard or calibrator as described elsewhere herein).

The incubation steps of a typical sandwich assay can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample, which is capable of being specifically bound. Further details, on how the measurement can be carried out, are also found elsewhere herein. The amount of formed complex shall be transformed into an amount of the biomarker reflecting the amount indeed present in the sample.

The term “amount” as used herein encompasses the absolute amount of total NT-proBNP or of unglycosylated NT-proBNP, the relative amount or concentration of the said total NT-proBNP or unglycosylated NT-proBNP, as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters, which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations. According to preferred embodiments of the subject invention, the determination of an “amount” is performed by the disclosed system, whereby a computing device determines the “amount” based on contacting and measuring steps performed by one or more analyzer units of said system.

In step c) of the method of the present invention, a score of the amounts determined in steps a) and b), i.e. the amount of total NT-proBNP and the amount of unglycosylated NT-proBNP is calculated.

The term “calculating” as used herein refers to assessing a score, which is based on the amount of total NT-proBNP and the amount of unglycosylated NT-proBNP determined in the sample(s) of the subject. For example, it is envisaged to calculate a score based on the amount of total NT-proBNP and the amount of unglycosylated NT-proBNP, i.e. a single score, and to compare this score to a reference score. The calculated score combines information on the amount of total NT-proBNP and the amount of unglycosylated NT-proBNP. Moreover, the biomarkers may be weighted in the score in accordance with their contribution to the establishment of the diagnosis. The score can be regarded as a classifier parameter for diagnosing atrial fibrillation. In particular, the score shall enable the diagnosis of AF based on the comparison with a reference score. The reference score is preferably a value, in particular a cut-off value, which allows for differentiating between a subject who suffers from AF and a subject who does not suffer from AF.

Preferably, the score is a ratio, i.e, a ratio of the amount of total NT-proBNP and the amount of unglycosylated NT-proBNP. Thus, the ratio calculated in step c) is compared to a reference ratio. In an embodiment, the ratio is the ratio of the amount of total NT-proBNP to the amount of unglycosylated NT-proBNP. In an alternative embodiment, the ratio is the ratio of the amount of unglycosylated NT-proBNP to the amount of total NT-proBNP.

In step d) of the method of the present invention, the score calculated in step c) shall be compared with a reference score. For example, a calculated ratio shall be compared to a reference ratio.

The term “comparing” as used herein encompasses comparing the score calculated for a sample from a test subject, which a suitable reference source specified elsewhere in this description. The comparison is, preferably, assisted by automation. For example, a suitable computer program comprising algorithms for the comparison of subject’s calculated score and the reference score may be used. Such computer programs and algorithms are well known in the art. Notwithstanding the above, a comparison can also be carried out manually. The computer program may further evaluate the result of the comparison, i.e. automatically provides the desired assessment in a suitable output format, i.e. the diagnostic result. The said diagnostic result may, preferably, serve as an aid for establishing the final diagnosis of atrial fibrillation by, e.g., a medical practitioner.

The calculation step and/or the comparison step may be carried out by using a computer comprising a processing unit.

Based on the comparison of the calculated score with the reference score, it shall be possible to assess whether the test subject suffers from atrial fibrillation, or not. For example, a result of a comparison may be given as raw data, and in some cases as an indicator in the form of a word, phrase, symbol, or numerical value which may be indicative of a particular diagnosis. Therefore, the reference score to be chosen so that either a difference or an identity of the calculated score to the calculated score allows for identifying those test subjects which belong into the group of subjects which suffer from atrial fibrillation, or not. The method allows either excluding (rule-out) or identifying (rule-in) a subject who is suffering from atrial fibrillation. Differences in the score, i.e. increases or decreases, as used herein, preferably, are differences which are statistically significant.

Preferably, the reference score, such as the reference ratio, shall allow for differentiating whether a subject suffers from atrial fibrillation, or not. Preferably, the diagnosis is made by assessing whether the score of the test subject is above or below the reference score It is not necessary to provide an exact reference score A relevant reference score can be obtained by correlating the sensitivity and specificity and the sensitivity/specificity for any score. A reference score resulting in a high sensitivity results in a lower specificity and vice versa.

In some embodiments, the reference score derived from a sample from a subject (or from samples group of subjects) known to suffer from atrial fibrillation.

In some embodiments, the reference score derived from a sample from a subject (or from samples group of subjects) known not to suffer from atrial fibrillation.

In the following, preferred diagnostic algorithms are provided.

As set forth above, the score calculated in step c) may be a ratio. In an embodiment, the ratio is the ratio of the amount of total NT-proBNP to the amount of unglycosylated NT-proBNP. In this case, a ratio (i.e. a calculated ratio) which is lower than the reference ratio is indicative for a subject who suffers from atrial fibrillation. A ratio which is larger than the reference ratio is indicative for a subject who does not suffer from atrial fibrillation.

In an alternative embodiment, the calculated ratio is the ratio of the amount of unglycosylated NT-proBNP to the amount of total NT-proBNP. In this case, a ratio (i.e. a calculated ratio) which is larger than the reference ratio is indicative for a subject who suffers from atrial fibrillation. A ratio which is lower than the reference ratio is indicative for a subject who does not suffer from atrial fibrillation.

in a preferred embodiment of method of the present invention, the method further comprises the step of recommending a suitable therapy, if atrial fibrillation has been diagnosed. Alternatively, the method further comprises the step of initiating a suitable therapy, if atrial fibrillation has been diagnosed.

The term “recommending” as used herein means establishing a proposal for a therapy which could be applied to the subject. However, it is to be understood that applying the actual therapy whatsoever is not comprised by the term. The therapy to be recommended depends on the out-come of the diagnosis provided by the method of the present invention. The recommendation step referred to above can also, preferably, be automated. Preferably, the diagnosis obtained by the method of the present invention, i.e. the diagnostic result of the method, will be used to search a database comprising recommendations of therapeutic measures for the individual possible diagnostic results.

In an embodiment, the therapy to be recommended or initiated is the administration of at least one anticoagulant, i.e. anti-coagulation therapy. Anticoagulation therapy is preferably a therapy which aims to reduce or prevent coagulation of blood and related stroke. In a preferred embodiment, the at least one anticoagulant is selected from the group consisting of heparin, a coumarin derivative (i e. a vitamin K antagonist), in particular warfarin or dicumarol, oral anticoagulants, in particular dabigatran, rivaroxaban or apixaban, tissue factor pathway inhibitor (TFPI), antithrombin III, factor IXa inhibitors, factor Xa inhibitors, inhibitors of factors Va and VIIIa and thrombin inhibitors (anti-IIa type). In some embodiments, at least one anticoagulant is selected from the group consisting of a direct factor Xa inhibitor, a direct thrombin inhibitor and a PAR-1 antagonist. Accordingly, it is envisaged that the subject takes at least one of the aforementioned medicaments (if diagnosed to suffer from atrial fibrillation).

In some embodiments, the anticoagulant is a direct factor Xa inhibitor, such as apixaban, rivaroxaban, darexaban or edoxaban. In some embodiments, the anticoagulant is a direct thrombin inhibitor such as dabigntran. In some embodiments, the anticoagulant is a PAR-1 antagonist such as voraparar or atopaxar.

In another embodiment, the therapy to be recommended or initiated is cardioversion. Thus, the patient may be subjected to cardioversion. Cardioversion a medical procedure by which a cardiac arrhythmia is converted to a normal rhythm using electricity or drugs.

In some embodiments, the cardioversion is electrical cardioversion, such as synchronized electrical cardioversion.

In some embodiments, the cardioversion is drug induced cardioversion Thus, at least one antiarrhythmic agent is administered. In some embodiments, the at least one antiarrhythmic agent is selected from amiodarone, flecainide, ibutilide, lidocaine, procainamide, propafenone, quinidine and tocainide,

Advantageously, it has been further shown that the ratio unglycosylated NT-proBNP / total-NTpro BNP was lower in reports of heart failure (see Examples). Thus, the score as referred to herein allows to differentiate atrial fibrillation versus heart failure as source of NT-proBNP elevation, Thus, it allows to differentiate atrial fibrillation versus heart failure.

The definitions and explanations given herein above preferably apply mutatis mutandis to the following methods of the present invention.

The methods of the present invention may be also carried out as computer-implemented inventions. In an embodiment, one or more steps, such as the comparison step and/or the calculation step are carried out by a computer comprising a processing unit (i.e. a computer). In another embodiment, all steps are carried by a computer comprising a processing unit.

Accordingly, the present invention relates to a computer-implemented method for diagnosing atrial fibrillation in subject, comprising
(a) receiving at a processing unit
   (a1) a value for the amount of total NT-proBNP in sample from the subject and
   (a2) a value for the amount of the amount of unglycosylated NT-proBNP in a sample from the subject,
(b) processing the values received in step (a) with the processing unit, wherein said processing comprises
   (b1) calculating a score of the values (a1) and (a2) received in step a),
   (b2) comparing the calculated score with a reference score and
(c) optionally providing the diagnosis via an output device, wherein said diagnosis is based on the results of step b).

In some embodiments, the processing unit is comprised by a computer.

In some embodiments, step b) further comprises retrieving, at the processing unit, from a memory a reference score, i.e. a reference score which is suitable for the diagnosis of AF.

In an embodiment of the methods of the present invention, information on the diagnosis (according to the last step of the methods of the present invention) is provided via a display, configured for presenting the assessment. Accordingly, information may be provided whether the subject suffers from atrial fibrillation, or not, as described elsewhere herein. Further, recommendations for suitable therapeutic can be displayed. As described elsewhere herein, various therapeutic measures may be recommended. In this case, the treatment option or treatment option(s) may be shown in the display

In an embodiment of the methods of the present invention, the methods may comprise the further step of transferring the information on the assessment of the methods of the present invention to the subject’s electronic medical records.

Alternatively, the assessment made in the last step of the methods of the present invention can be printed by a printer. The print-out shall contain information on whether the patient is at risk, or not at risk and/or a recommendation of a suitable therapeutic measure.

The present invention further relates to i) the use of total NT-proBNP and unglycosylated NT-proBNP as biomarkers, or to ii) the use of at least one agent which specifically binds to unglycosylated NT-proBNP and of at least one agent which specifically binds to total NT-proBNP, for diagnosing atrial fibrillation. Preferably, said use in an in vitro use, i.e. is carried out in sample from the subject.

Preferred agents are disclosed elsewhere herein (such as antibodies, or antigen binding fragments thereof which bind to certain epitopes within NT-proBNP).

Finally, the present invention relates to a kit comprising at least one agent which specifically binds to unglycosylated NT-proBNP and at least one agent which specifically binds to total NT-proBNP.

The term “kit” as used herein refers to a collection of the aforementioned means, for example, provided in separately or within a single container. The container may comprise instructions for carrying out the method of the present invention.

### LIST OF EMBODIMENTS

In the following, preferred embodiments are summarized. The definitions and explanations given herein above preferably apply mutatis mutandis to the following embodiments.
1. A method for diagnosing atrial fibrillation in a subject, said method comprising the steps of
   a) determining the amount of total NT-proBNP in sample from the subject,
   b) determining the amount of unglycosylated NT-proBNP in a sample from the subject,
   c) calculating a score of the amounts determined in steps a) and b),
   d) comparing the calculated score with a reference score, and
   e) diagnosing atrial fibrillation in a subject.
2. The method of embodiment 1, wherein the sample is blood, serum, or plasma sample.
3. The method of embodiments 1 and 2, wherein the subject is a human subject.
4. The method of any one of embodiments 1 to 3, wherein the subject is suspected to suffer from atrial fibrillation.
5. The method of embodiment 4, wherein the subject who is suspected to suffer from atrial fibrillation has a history of atrial fibrillation.
6. The method of any one of embodiments 1 to 5, wherein unglycosylated NT-proBNP is unglycosylated at one or more positions selected from group consisting of T36, S37, S44, T48, S53, T58 and/or T71 of human NT-proBNP.
7. The method of embodiment 6, wherein unglycosylated NT-proBNP is unglycosylated at least at position S44.
8. The method of any one of embodiments 1 to 7, wherein the determination of the amount of unglycosylated NT-proBNP comprises contacting the sample with an antibody, or antigen-binding fragment thereof, which specifically detects unglycosylated NT-proBNP.
9. The method of embodiment 8, wherein the epitope of the antibody, or antigen-binding fragment thereof, comprises amino acid residues 42 to 46 of human NT-proBNP.
10. The method of any one of embodiments 1 to 9, wherein amount of total NT-proBNP is the amount of glycosylated and unglycosylated NT-proBNP.
11. The method of any one of embodiments 1 to 10, wherein the determination of the amount of total NT-proBNP comprises contacting the sample with an antibody, or antigen-binding fragment thereof, which specifically detects total NT-proBNP.
12. The method of embodiment 11, wherein the antibody, or antigen-binding fragment thereof, which specifically detects total NT-proBNP binds to a region of human NT-proBNP which does not carry an O-glycosylation site.
13. The method of embodiment 12, wherein the antibody, or antigen-binding fragment thereof, binds to an epitope present with the first 35 amino acids of NT-proBNP.
14. The method of embodiment 13, epitope of the antibody, or antigen-binding fragment thereof, comprises amino acid residues 13 to 16 of human NT-proBNP.
15. The method of any one of embodiments 1 to 14, wherein the score is a ratio.
16. The method of embodiment 15, wherein the ratio is the ratio of the amount of total NT-proBNP to the amount of unglycosylated NT-proBNP.
17. The method of embodiment 16, wherein a ratio which is lower than the reference ratio is indicative for a subject who suffers from atrial fibrillation.
18. The method of embodiment 15, wherein the ratio is the ratio of the amount of unglycosylated NT-proBNP to the amount of total NT-proBNP.
19. The method of embodiment 18, wherein a ratio which is larger than the reference ratio is indicative for a subject who suffers from atrial fibrillation.
20. The method of any one of embodiments 1 to 19, wherein atrial fibrillation is paroxysmal or persistent atrial fibrillation.
21. A computer-implemented method for diagnosing atrial fibrillation in subject, comprising
   (a) receiving at a processing unit
      (a1) a value for the amount of total NT-proBNP in sample from the subject and
      (a2) a value for the amount of the amount of unglycosylated NT-proBNP in a sample from the subject,
   (b) processing the values received in step (a) with the processing unit, wherein said processing comprises
      (b1) calculating a score of the values (a1) and (a2) received in step a),
      (b2) comparing the calculated score with a reference score and
   (c) optionally providing the diagnosis via an output device, wherein said diagnosis is based on the results of step b).
22. Use of i) total NT-proBNP and unglycosylated NT-proBNP as biomarkers, or the use of ii) at least one agent which specifically binds to unglycosylated NT-proBNP and of at least one agent which specifically binds to total NT-proBNP, for diagnosing atrial fibrillation.
23. A kit comprising at least one agent which specifically binds to unglycosylated NT-proBNP and at least one agent which specifically binds to total NT-proBNP.

The Figures show:

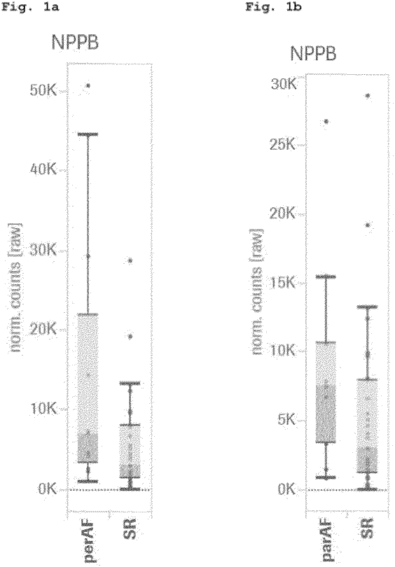

FIG. 1 Differential expression of NPPB in right atrial appendage tissue: A) persistent Aftb; B) paroxysmal Afib.

FIG. 2 Overview on O-glycosylation sites in proBNP epitopes. The sequence of human NT-proBNP is underlined twice. In the studies underlying the present invention, two antibodies were used: one antibody binding to epitope aa13-16 of NT-proBNP (for total NT-proBNP) and one antibody binding to epitope aa42-46 of NT-proBNP for NT-proBNP, i.e. unglycosylated NT-proBNP Other antibodies binding to other epitopes can be used as well, for example, the antibodies from Hytest which bind to epitopes as shown in the Figure. The antibodies are also disclosed in US20090163415A1.

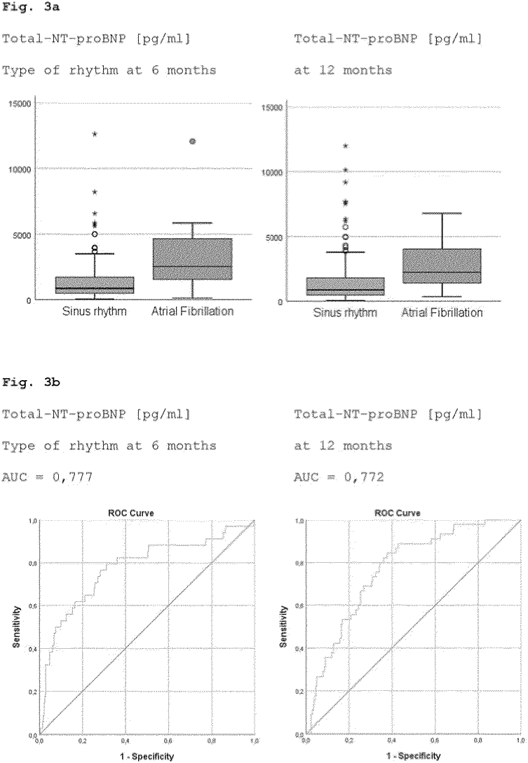

FIG. 3: Differentiation between patients with ongoing atrial fibrillation and patients in sinus rhythm based on total-NT-proBNP

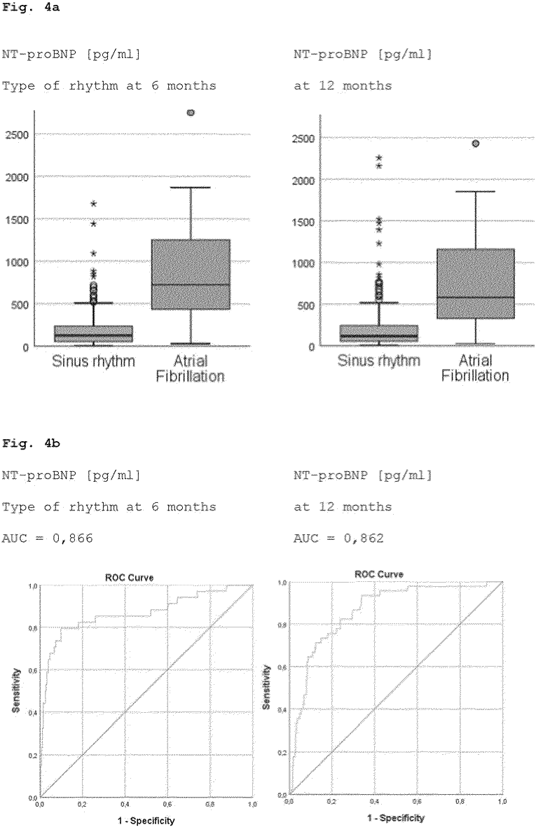

FIG. 4: Differentiation between patients with ongoing atrial fibrillation and patients in sinus rhythm based on unglycosylated NT-proBNP

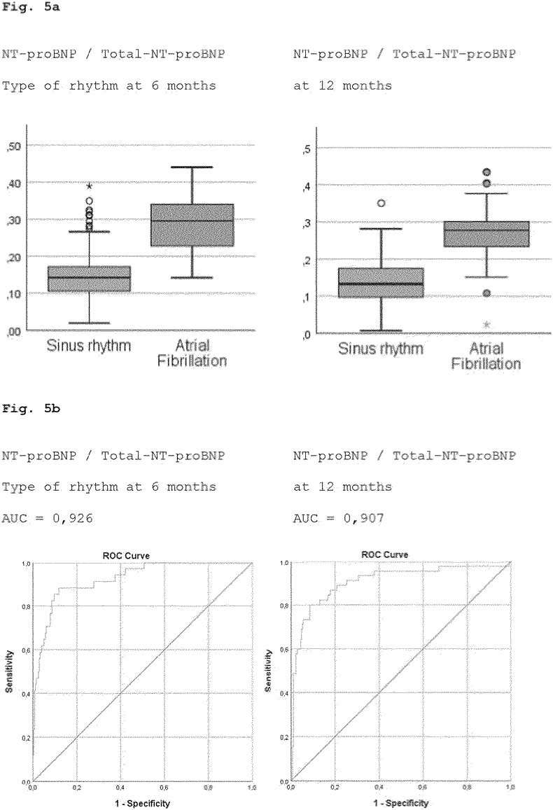

FIG. 5: Differentiation between patients with ongoing atrial fibrillation and patients in sinus rhythm based on ratio [unglycosylated NT-proBNP) / [total-NT-proBNP].

### EXAMPLES

The invention will be merely illustrated by the following Examples. The said Examples shall, whatsoever, not be construed in a manner limiting the scope of the invention.

### Example 1: Differential Expression of Human NPPB in Right Atrial Appendage Tissue (Mapping Study)

Right atrial appendage tissue was sampled during open chest surgery because of CABG or valve surgery. Evidence of AF or SR (controls, sinus rhythm) was generated during surgery with simultaneous Endo-Epicardial High Density Activation Mapping. Tissue samples were taken during surgery. Patients with AF and controls were matched with regard to gender, age and comorbidities.

Atrial tissue samples were prepared for
paroxysmal AF patients; n=14 patients persistent AF patients; n=8 patients
control patients in SR; n=27 patients.

Differential expression of NPPB was determined in RNAseq analyses applying the algorithms RSEM and DESEQ2. The results are shown in FIG. 1 [A) persistent Afib B) paroxysmal Afib].

As shown in FIG. 1, NPPB expression was found to be upregulated in the analyzed right atrial appendage tissues of the patients with paroxysmal and persistent AF versus the control patients in sinus rhythms. The highest levels were observed in patients with persistent AF. These data support the hypothesis of atrial appendage dependency of NT-proBNP and its increase with disease severity.

### Example 2. Detection of NT-proBNP

The amounts of total-NT-proBNP and NT-proBNP were measured by sandwich assays. Total NT-proBNP was determined by using an antibody against amino acids 13 to 16 of NT-proBNP as capture antibody (monoclonal antibody 17.3.1). NT-proBNP was determined by the using an antibody against amino acids 42 to 46 of NT-proBNP as capture antibody (monoclonal antibody 1.21.03) using the Roche Elecays® proBNP 11 NT-proBNP assay following the manufacturer’s instructions. This antibody detects NT-proBNP which is not O-glycosylated position S44.

For both assays, a detection antibody was used which binds to amino acids 27 to 31 (monoclonal antibody 18.4.34, see FIG. 2).

The epitopes are shown in FIG. 2.

Higher values generated with non-glycosylated total-NT-proBNP assay due to elimination of effects caused by O-Glycosylation blocking detection at position S44. In clinical setting this invention increases the sensitivity, where detection of different entities causes improvement in specificity and the sum in sensitivity.

A ratio between total NT-proBNP and unglycosylated NT-proBNP can be formed which allows normalization.

### Example 3: Detection of Atrial Fibrillation in the Biomarker Sub-Study of the GISSI-AF Trial

In the biomarker sub-study of the GISSI-AF trial, blood samples were collected at study entry, and after 6 and 12 months of follow-up. For more details on the GISSI-AF trial, see the main publication: GISSI-AF Investigators, New Engl J Meet 2009;360:1606-17. For more details on the biomarker substudy, see: Latini R et al., J Intern Meet 2011;269-160-71

For 382 patients total-NT-proBNP and NT-proBNP values from plasma samples were obtained at baseline. After 24 weeks, out of 360 patients 38 developed paroxysmal atrial fibrillation. After 52 weeks, 48 out of 357 have developed atrial fibrillation.

NT-proBNP and total NT-proBNP was determined in the atrial fibrillation sub-cohort selected from the GISSI AF study. Elevated circulating NT-proBNP and total NT-proBNP levels were observed in samples from subjects with on-going atrial fibrillation versus controls.

TABLE 1

Association with Prevalent Atrial Fibrillation:

| | Baseline | 24 weeks | 12 months |
|---|---|---|---|
| Sinus Rhythm | 382 (100%) | 322 | 309 (86.6%) |
| Atrial Fibrillation | 0 | 38 | 48 (13.4%) |

The sample size considered is composed of 382 patients with measurements of NTproBNP at baseline. The variable SR (sinus rhythm) vs AF (atrial fibrillation) was recorded from variable “ritmo all′ECG″” in the CRF.

Conclusion: Measurement of total-NT-proBNP and NT-proBNP showed upon analysis that combination of both parameters results in far better diagnostic value than each single marker.

TABLE 2

Biomarker concentrations and biomarker ratio and atrial fibrillation/sinus rhythm recorded by ECG during scheduled visits at 6 and 12 months

| Biomark er | Time | | N | Median | AUC | 95 %Cl | P |
|---|---|---|---|---|---|---|---|
| total NT-proBNP pg/mL | 6 months | Sinus Rhythm | 280 | 865 | 0.78 | (0.68-0.87) | < 0.0001 |
| | | Atrial Fibrillation | 34 | 2532 | | | |
| | 12 months | Sinus Rhythm | 268 | 878 | 0.77 | (0.70-0.84) | < 0.0001 |
| | | Atrial Fibrillation | 45 | 2241 | | | |
| NT-proBNP pg/mL | 6 months | Sinus Rhythm | 284 | 128.0 | 0.87 | (0.78-0.95) | < 0.0001 |
| | | Atrial Fibrillation | 34 | 724.5 | | | |
| | 12 months | Sinus Rhythm | 276 | 116.5 | 0.86 | (0.81-0.92) | < 0.0001 |
| | | Atrial Fibrillation | 45 | 580.0 | | | |
| Ratio NT-proBNP / total-NTpro BNP | 6 months | Sinus Rhythm | 280 | 0.14 | 0.93 | (0.88-0.97) | < 0.0001 |
| | | Atrial Fibrillation | 34 | 0.3 | | | |
| | 12 months | Sinus Rhythm | 268 | 0.13 | 0.91 | (0.85-0.96) | < 0.0001 |
| | | Atrial Fibrillation | 45 | 0.28 | | | |

To assess the strength of the association between studied biomarkers and ongoing AF (i.e. biomarkers assayed while an AF rhythm was recorded), ROC analyses were done. At 6 and 12 months for the ratio NT-proBNP/ total NT-proBNP the observed AUC was higher compared with the AUCs for each biomarker alone : ratio NTproBNP/ total NTproBNP (AUC6m=0.93 and AUC12m=0.91), total NTproBNP (AUC6M=0.78 and AUC12M=0.77), NT-proBNP (AUC6M=0.87 and AUC12M--0.86).

It can be not in the table 2 and in the FIGS. 3, 4 and 5, that the ratio of NT-proBNP/ total NT-proBNP proved to be better in the prediction of prevalent / ongoing AF than either one of the natriuretic peptides alone.

It was further observed in the GISSI AF study that the ratio NT-proBNP/total NT-proBNP was associated with incident / recurrent AF (p= 0.058).

In conclusion in patients with ongoing AF a lower degree of glycosylation of NT-proBNP has been found versus patients in sinus rhythm. Determination of the ratio NTproBNP/total NTproBNP is suitable to further improve the diagnostic accuracy versus each of the natriuretic peptides alone.

TABLE 3

Biomarker concentrations and biomarker ratio and atrial fibrillation/sinus rhythm recorded by ECG during scheduled visits at 6 and 12 months in patients divided by gender

| Biomarker | Gender | Time | | N | AUC | 95%Cl | P |
|---|---|---|---|---|---|---|---|
| Ratio NT-proBNP /total-NTpro BNP | Females | 6 months | Sinus Rhythm | 100 | 0.94 | (0.87-0.99) | < 0.0001 |
| | | | Atrial Fibrillation | 11 | | | |
| | | 12 months | Sinus Rhythm | 96 | 0.95 | (0.90-0.99) | < 0.0001 |
| | | | Atrial Fibrillation | 11 | | | |
| Ratio NT-proBNP /total-NTpro BNP | Males | 6 months | Sinus Rhythm | 180 | 0.92 | (0.86-0.97) | < 0.0001 |
| | | | Atrial Fibrillation | 23 | | | |
| | | 12 months | Sinus Rhythm | 172 | 0.92 | (0.86-0.97) | < 0.0001 |

To assess the strength of the association between studied biomarkers and ongoing AF in female patients versus male patients (i.e. biomarkers assayed while an AF rhythm was recorded), ROC analyses were done separately for male and female study participants. At 6 and 12 months for the ratio NT-proBNP/ total NT-proBNP the observed AUC was higher in female patients as compared with male patients the ratio NTproBNP/ total NTproBNP in female patients (AUC6m-0.94 and AUC12m-0.95), the ratio NTyroBNP/total NTproBNP in male patients (AUC6M=0.92 and AUC12M=0.92).

As shown in the table 3 the ratio of NTproBNP/total NT-proBNP was found to be particularly well performing in the detection of ongoing AF in the subgroup of female participants of the study. While the Ratio NT-proBNP / total-NTpro BNP was ∼0.15 in Afib (Table 2), but higher in reports of heart failure (0.19-0.31; Vodovar N et al European Heart Journal (2014) 35, 3434-3441), the present invention of using the ratio of NT-proBNP / total-NTpro BNP in AFib allows to better differentiate AFib versus HF as source of NT-proBNP elevation, and thus to better differentiate AFib vs heart failure.

In conclusion for the detection of AF in females the ratio NTproBNP/total NTpro-BNP may be very useful

The GISSI AF study comprises patients with moderate severe comorbidities: clinically diagnosed HF or LVEF<40% was low (11%), history of stroke (4%), diabetes (13 %), history of hypertension (84 %), documented CAD (11 %). The incidence of AF was not significantly different in patients with any of the observed comorbidities versus patients without respective comorbidities.

In conclusion, patients with incident AF or with a history of AF were found to have a lower extent of glycosylation of NTproBNP as compared to patients in sinus rhythm.

Overall it was observed that the ratio NTproBNP/total NTproBNP allows for an improved diagnosis of prevalent AF in patients with a history of AF or other risk factors for incident AF recurrence.

1. A method for diagnosing atrial fibrillation in a subject, said method comprising
determining an amount of total NT-proBNP in a sample from the subject,
determining an amount of unglycosylated NT-proBNP in the sample from the subject,
calculating a score of the amounts of the total NT-proBNP and the unglycosylated NT-proBNP,
comparing the calculated score with a reference score, and
diagnosing atrial fibrillation in the subject based on the comparison of the calculated score with the reference score.

2. The method of claim 1, wherein the sample is a blood, a serum, or a plasma sample, and/or
wherein the subject is a human subject.

3. The method of claim 1, wherein the subject is suspected to suffer from atrial fibrillation.

4. The method of claim 1, wherein unglycosylated NT-proBNP is unglycosylated at one or more positions selected from T36, S37, S44, T48, S53, T58, and/or T71 of human NT-proBNP.

5. The method of claim 4, wherein unglycosylated NT-proBNP is unglycosylated at least at position S44.

6. The method of claim 1, wherein the determination of the amount of unglycosylated NT-proBNP comprises contacting the sample with an antibody, or antigen-binding fragment thereof, which specifically detects unglycosylated NT-proBNP.

7. The method of claim 6, wherein the epitope of the antibody, or antigen-binding fragment thereof, comprises amino acid residues 42 to 46 of human NT-proBNP.

8. The method of claim 1, wherein the amount of total NT-proBNP is the amount of glycosylated and unglycosylated NT-proBNP.

9. The method of claim 8, wherein the antibody, or antigen-binding fragment thereof, which specifically detects total NT-proBNP, binds to a region of human NT-proBNP which does not carry an O-glycosylation site.

10. The method of claim 9,
wherein the antibody, or antigen-binding fragment thereof, binds to an epitope present within the first 35 amino acids of NT-proBNP, and
wherein the epitope of the antibody, or antigen-binding fragment thereof, comprises amino acid residues 13 to 16 of human NT-proBNP.

11. The method of claim 1, wherein the calculated score is a ratio, and
wherein the ratio is the ratio of the amount of total NT-proBNP to the amount of unglycosylated NT-proBNP, wherein a ratio which is lower than the reference ratio is indicative for a subject who suffers from atrial fibrillation, or
wherein the ratio is the ratio of the amount of unglycosylated NT-proBNP to the amount of total NT-proBNP, wherein a ratio which is larger than the reference ratio is indicative for a subject who suffers from atrial fibrillation.

12. The method of claim 1, wherein atrial fibrillation is paroxysmal or persistent atrial fibrillation.

13. A computer-implemented method for diagnosing atrial fibrillation in a subject, comprising
receiving at a processing unit
a value for an amount of total NT-proBNP in a sample from the subject, and
a value for an amount of unglycosylated NT-proBNP in the sample from the subject:
processing the values for total NT-proBNP and unglycosylated NT-proBNP with the processing unit, wherein said processing comprises
calculating a score of the values for total NT-proBNP and unglycosylated NT-proBNP, and
comparing the calculated score with a reference score; and
providing the diagnosis via an output device, wherein said diagnosis is based on processing the values for total NT-proBNP and unglycosylated NT-proBNP.

14. (canceled)

15. A kit comprising at least one agent which specifically binds to unglycosylated NT-proBNP and at least one agent which specifically binds to total NT-proBNP.

16. The method of claim 8, wherein the determination of the amount of total NT-proBNP comprises contacting the sample with an antibody, or antigen-binding fragment thereof, which specifically detects total NT-proBNP.

17. The method of claim 6, wherein the antibody is the monoclonal antibody MAB 1.21.3, or an antigen-binding fragment thereof.

18. The method of claim 16, wherein the epitope of the antibody, or antigen-binding fragment thereof, comprises amino acid residues 13 to 16 of human NT-proBNP.

19. The method of claim 16, wherein the antibody is the monoclonal antibody MAB 17.3.1, or an antigen-binding fragment thereof.

20. The method of claim 6, wherein the antibody, or antigen-binding fragment thereof, is used in a sandwich assay as a capture antibody in combination with at least one other antibody that binds to an epitope of NT-proBNP which is not glycosylated.

21. The method of claim 3, wherein the subject who is suspected to suffer from atrial fibrillation has a history of atrial fibrillation.


SEQUENCE LISTING (2 sequences)

SEQ ID NO: 1 - protein, 76 aa, homo sapiens
HPLGSPGSASDLETSGLQEQRNHLQGKLSELQVEQTSLEPLQESPRPTGVWKSREVATEGIRGHRKMVLYTLRAPR

SEQ ID NO: 2 - protein, 134 aa, homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH